(19) United States
(12) Patent Application Publication
(10) Pub. No.: US 2018/0002737 A1
(43) Pub. Date: Jan. 4, 2018

(54) NUCLEIC ACID AMPLIFICATION METHOD AND NUCLEIC ACID AMPLIFICATION APPARATUS

(71) Applicant: Seiko Epson Corporation, Tokyo (JP)

(72) Inventor: Masayuki UEHARA, Gifu (JP)

(21) Appl. No.: 15/543,166

(22) PCT Filed: Dec. 15, 2015
(86) PCT No.: PCT/JP2015/006236

§ 371 Date: Jul. 12, 2017

(30) Foreign Application Priority Data
Jan. 14, 2015 (JP) 2015-004833

(51) Int. Cl. C12Q 1/68; B01L 7/00; C12M 1/00

(52) U.S. Cl. CPC C12Q 1/686; C12Q 1/6853; B01L 7/5255; B01L 2300/18; C12Q 1/6806; C12M 1/00

(57) ABSTRACT

A nucleic acid amplification method includes a step of heating a first region of a container housing a droplet containing a target nucleic acid and a sample necessary for amplification of the target nucleic acid to a denaturation temperature of the target nucleic acid and heating a second region different from the first region to a synthesis temperature of the target nucleic acid, and an amplification step of repeating a cycle through a denaturation stage at which the droplet housed in the container is moved to and retained in the first region and a synthesis stage at which the droplet is moved to and retained in the second region at a plurality of times. At the amplification step, periods of part of cycles of the plurality of cycles are made shorter than periods of the other cycles.

### TECHNICAL FIELD

This invention relates to a nucleic acid amplification method and a nucleic acid amplification apparatus.

### BACKGROUND ART

The PCR (polymerase chain reaction) method is a technique of amplifying nucleic acids including DNA (deoxyribonucleic acid) by repeatedly providing temperature changes to the nucleic acids using denaturation and differences in annealing of the nucleic acids caused by differences in chain length in the nucleic acids.

As a nucleic acid amplification apparatus using the PCR method, a PCR apparatus of JP-A-2012-115208 was proposed by the applicant (see e.g. JP-A-2012-115208). In a biochip attached to the PCR apparatus of JP-A-2012-115208, a channel in which a reaction solution containing a target nucleic acid moves is formed, and the reaction solution is housed in the channel and the channel is filled with a liquid having smaller specific gravity than the reaction liquid and not miscible in the reaction solution.

In the PCR apparatus, in the case where a biochip is attached to an attachment part for attachment of the biochip, a heating part that heats a first region of the channel formed in the biochip and a heating part that heats a second region at a different temperature from that for the first region are provided. Further, in the PCR apparatus, a drive mechanism that switches the arrangement of the attachment part and the heating parts between a first arrangement and a second arrangement is provided. By the drive mechanism, the reaction solution of the biochip attached to the attachment part is moved to each other between the first region and the second region heated to the different temperatures from each other. According to the PCR apparatus of JP-A-2012-115208, the amplification reaction period may be shortened compared to the case where the temperature of the whole biochip is switched between different temperatures from each other.

### SUMMARY OF INVENTION

### Technical Problems

Now, further shortening of the production time of the PCR product in the above described PCR apparatus is required. However, if the amplification reaction period (cycle time) is made too short, significant reduction of the amplification efficiency is concerned.

Accordingly, an object of the invention is to provide a nucleic acid amplification method and a nucleic acid amplification apparatus that may shorten a production time of a PCR product while suppressing reduction of amplification efficiency.

### Solution to Problems

In order to achieve the object, a nucleic acid amplification method of the invention includes a heating step of heating a first region of a container housing a droplet containing a target nucleic acid and a sample necessary for amplification of the target nucleic acid to a denaturation temperature of the target nucleic acid and heating a second region independent of the first region to a synthesis temperature of the target nucleic acid, and an amplification step of repeating a cycle through a denaturation stage at which the droplet housed in the container is moved to and retained in the first region and a synthesis stage at which the droplet is moved to and retained in the second region at a plurality of times, wherein, at the amplification step, periods of part of cycles of the plurality of the cycles are made shorter than periods of the other cycles.

Further, a nucleic acid amplification apparatus of the invention includes an attachment part to which a container housing a droplet containing a target nucleic acid and a sample necessary for amplification of the target nucleic acid is attached, a heater that heats a first region in the container attached to the attachment part to a denaturation temperature of the target nucleic acid and heating a second region independent of the first region to a synthesis temperature of the target nucleic acid, a movement mechanism that moves the droplet from the first region to the second region or from the second region to the first region without changing relative positions of the container attached to the attachment part and the heater, and a control section that controls the movement mechanism to repeat a cycle through a denaturation stage at which the droplet is retained in the first region and a synthesis stage at which the droplet is retained in the second region at a plurality of times, wherein the control section makes periods of part of cycles of the plurality of the cycles shorter than periods of the other cycles.

### BRIEF DESCRIPTION OF DRAWINGS

FIG. 1 shows a section of a cartridge.

FIG. 2 shows a state of a tank before attached to a cartridge main body.

FIG. 3 shows a state in which a specimen is introduced into the tank.

FIGS. 4A and 4B show states of the cartridge before and after pushed into from outside.

FIGS. 5A and 5B show the cartridge with a focus on a PCR container therein.

FIG. 6 is a block diagram of a nucleic acid amplification apparatus.

FIGS. 7A and 7B show states of a rotation mechanism.

FIGS. 8A and 8B show a state in which the cartridge has been attached to an attachment part.

FIGS. 9A-9D show states of thermal cycling processing.

FIG. 10 schematically shows temperature shifts of a droplet.

FIG. 11 is a flowchart showing a thermal cycling processing procedure of a control section.

FIG. 12 schematically shows temperature shifts of a droplet in the second embodiment.

FIGS. 13A and 13B schematically show temperature shifts of a droplet (1) in other examples than the embodiments.

FIG. 14 schematically shows temperature shifts of a droplet (2) in another example than the embodiments.

### DESCRIPTION OF EMBODIMENTS

As below, embodiments for implementing the invention will be exemplified using the accompanying drawings. The embodiments exemplified as below are for facilitation of understanding of the invention, but not for limited interpretation of the invention. Changes and improvements may be made without departing from the scope of the invention.

(1) First Embodiment

As the first embodiment, first, a cartridge to be attached to a nucleic acid amplification apparatus will be explained, and then, the nucleic acid amplification apparatus and a method therefor will be explained.

Cartridge

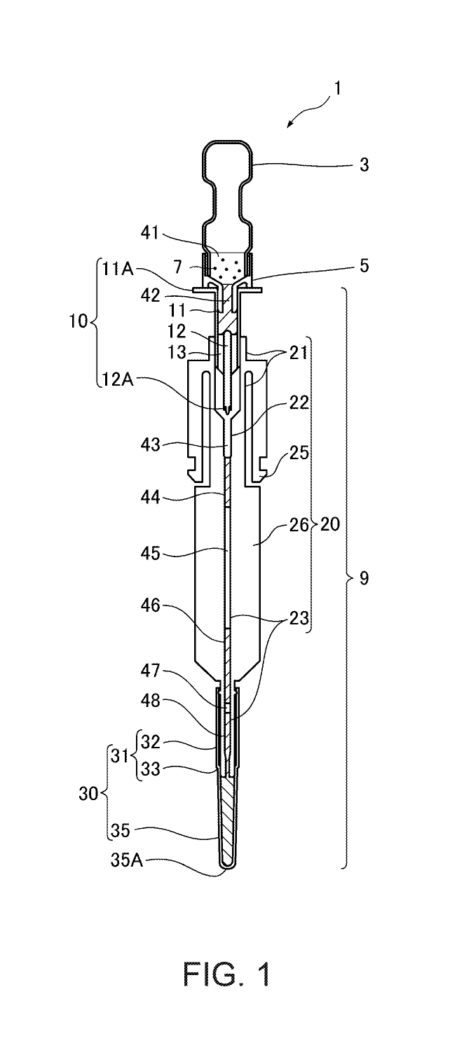

FIG. 1 shows a section of a cartridge 1. As shown in FIG. 1, the cartridge 1 has a tank 3, an adapter 5, and a cartridge main body 9, and the tank 3 and the cartridge main body 9 are adapted to be detachable using the adapter 5.

<Tank>

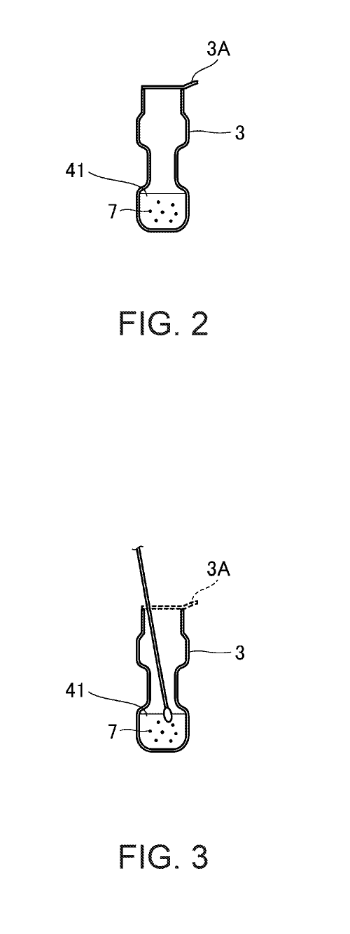

FIG. 2 shows a state of the tank 3 before attached to the cartridge main body 9. As shown in FIG. 2, the tank 3 is a container into which a specimen is introduced. An opening is formed in a predetermined part of the tank 3, and a sealing member 3A is provided in the opening.

Within the tank 3, a dissolution and adsorption solution 41 for extracting nucleic acids of cells originating from a living organism such as human or bacteria, or virus, and magnetic beads 7 as solid-phase supports having binding properties to the nucleic acids are housed.

The dissolution and adsorption solution 41 may be a buffer solution and preferably neutral from pH 6 to 8. The dissolution and adsorption solution 41 contains a chaotropic agent. The chaotropic agent is not particularly limited as long as the agent has a function of producing chaotropic ions (monovalent anions having large ion radii) in a water solution and increasing water solubility of hydrophobic molecules and contributes to adsorption of nucleic acids to the magnetic beads 7. Specific examples include guanidine thiocyanate, guanidine hydrochloride, sodium iodide, potassium iodide, sodium perchlorate, etc.

Further, the dissolution and adsorption solution 41 may contain surfactant for breakdown of cell membranes or denaturation of protein contained in cells. The surfactant is not particularly limited as long as the surfactant is generally used for nucleic acid extraction from cells or the like. Specific examples include triton surfactants such as Triton-X, non-ionic surfactants such as Tween 20, anionic surfactants such as sodium N-lauroylsarcosine (SDS). Furthermore, the dissolution and adsorption solution 41 may contain a reducing agent such as 2-mercaptoethanol or dithiothreitol.

An example of the composition of the dissolution and adsorption solution 41 includes 5M guanidine thiocyanate, 2% Triton X-100, 50 mM Tris-HCl (pH 7.2).

FIG. 3 shows a state in which a specimen is introduced into the tank 3. As shown in FIG. 3, when a specimen is introduced into the tank 3, the sealing member 3A is removed from the tank 3 and the opening of the tank 3 is opened, and the specimen collected by a collecting tool such as a cotton swab is introduced into the tank 3 from the opening of the tank 3.

When the specimen contains e.g. virus, the envelope and the capsid of the virus are dissolved by the dissolution and adsorption solution 41 and virus nucleic acids are released and adsorbed to the surfaces of the magnetic beads 7.

The adapter 5 is fitted into the opening of the tank 3 and the opening of the tank 3 is allowed to communicate with the cartridge main body 9 via the adapter 5 (see FIG. 1).

<Cartridge Main Body>

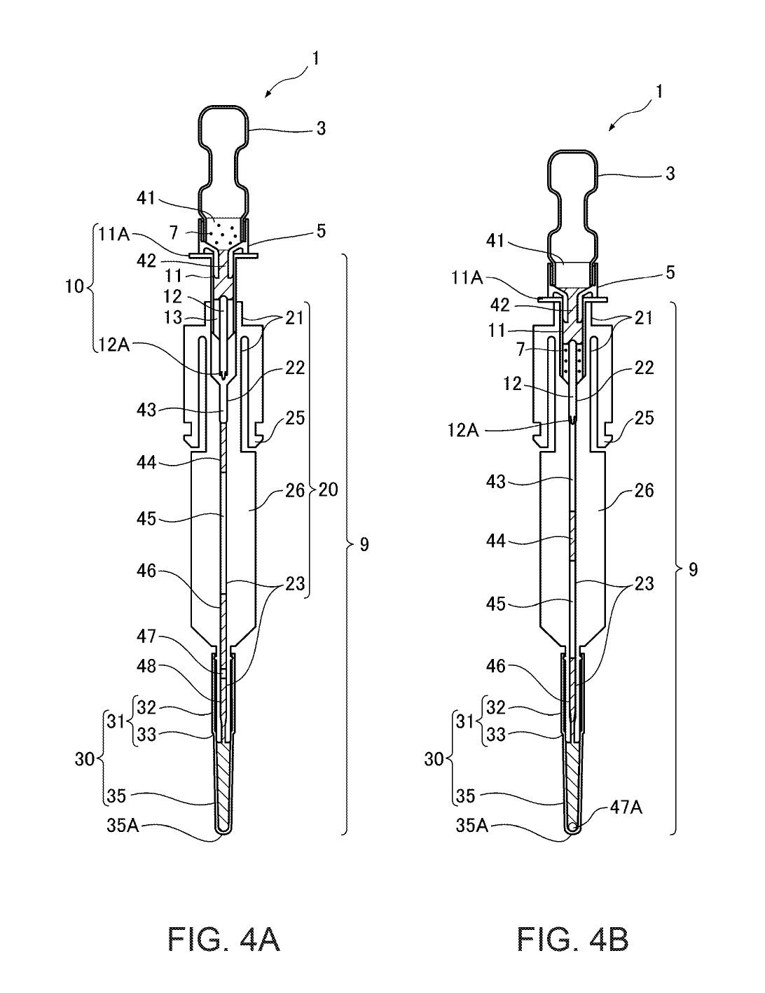

FIG. 4 shows states of the cartridge 1 before and after a plunger 10 is pushed into a syringe 32. Specifically, FIG. 4(A) shows the state of the cartridge 1 before the plunger 10 is pushed into and FIG. 4(B) shows the state of the cartridge 1 after the plunger 10 is pushed into.

As shown in FIGS. 4A and 4B, the cartridge main body 9 has the plunger 10, a tube 20, and a PCR container 30.

<<Plunger>>

The plunger 10 is a pusher that pushes a predetermined amount of liquid within the tube 20 serving as the syringe out of the terminal end on the tube 20 side into the PCR container 30, and has a cylindrical portion 11 and a rod-like portion 12. The cylindrical portion 11 and the rod-like portion 12 may be integrally formed or separately formed.

The cylindrical portion 11 is a part communicating with the opening of the tank 3 via the adapter 5. A flange-shaped mount 11A protruding outwards from the cylindrical portion 11 toward the outside of the opening is formed around the opening of the cylindrical portion 11 on the tank 3 side. The adapter 5 attached to the mount 11A is fitted into the opening of the cylindrical portion 11, and thereby, the opening of the cylindrical portion 11 and the opening of the tank 3 communicate. Note that the mount 11A is a pressed part to be pressed by the nucleic acid amplification apparatus.

The end of the cylindrical portion 11 on the tube 20 side is fitted in the inner wall of an upper syringe 21 of the tube 20 and slidable along the upper syringe 21 while being in contact with the inner wall of the upper syringe 21. Note that a distance between the mount 11A of the plunger 10 and the upper edge of the tube 20 is a slide length of the plunger 10.

The rod-like portion 12 is supported by a rib 13 projecting from the inner wall of the end of the cylindrical portion 11 on the tube 20 side. The end of the cylindrical portion 11 on the tube 20 side is located inside of the upper syringe 21 apart from a lower syringe 22 in an initial state in which the plunger 10 is not pushed (see FIG. 4(A)). On the other hand, when plunger 10 is pushed, the end of the cylindrical portion 11 on the tube 20 side is inserted into the lower syringe 22 of the tube 20 and slides along the lower syringe 22 in contact with the inner wall of the lower syringe 22 (see FIG. 4(B)).

A seal 12A is formed on the tip end of the rod-like portion 12 on the tube 20 side, and the backflow of the liquid within the tube 20 into the plunger 10 is prevented by the seal 12A. Note that the liquid within the tube 20 is pushed outside to the downstream side by an amount of the volume of the seal 12A sliding within the lower syringe 22.

Inside of the plunger 10, an oil 42 that phase-separates from the other solution and a first cleaning solution 43 having larger specific gravity than the oil 42 are housed. The oil 42 within the plunger 10 has smaller specific gravity than the first cleaning solution 43. Accordingly, when the cartridge main body 9 is stood with the mount 11A of the plunger 10 up, as shown in FIG. 4(A), the oil 42 is placed between the dissolution and adsorption solution 41 within the tank 3 and the first cleaning solution 43 of the cartridge main body 9.

Note that the oil 42 includes e.g. 2CS silicone oil and the first cleaning solution 43 includes e.g. 8M guanidine hydrochloride, 0.7% Triton X-100. Further, when the first cleaning solution 43 contains the above described chaotropic agent, cleaning can be performed while the adsorption state of the nucleic acid adsorbed to the magnetic beads 7 is maintained or strengthened.

<<Tube>>

The tube 20 has the upper syringe 21, the lower syringe 22, and a capillary 23, and the inner diameters of the respective portions are gradually smaller from the plunger 10 toward the PCR container 30.

The upper syringe 21 is a cylindrical part that functions as a syringe for the cylindrical portion 11 of the plunger 10, and, as described above, the cylindrical portion 11 of the plunger 10 is slidably in contact with the inner wall of the upper syringe 21.

The lower syringe 22 is a cylindrical part that functions as a syringe for the rod-like portion 12 of the plunger 10, and, as described above, the seal 12A of the rod-like portion 12 of the plunger 10 is slidably fitted in the inner wall of the lower syringe 22.

The capillary 23 is a canalicular part having a plurality of kinds of liquids. The end of the capillary 23 on the PCR container 30 side is inserted into the PCR container 30, and the tip end of the inserted portion is tapered to be thinner. That is, the inner diameter of the terminal end of the capillary 23 (the opening diameter of the capillary 23) is smaller than the inner diameter of the other parts than the terminal end of the capillary 23.

Within the capillary 23, a first oil plug 44, a cleaning solution plug 45, a second oil plug 46, a reaction solution plug 47, and a third oil plug 48 are sequentially placed from the plunger 10 side.

“Plug” refers to a liquid occupying a specific one section within the capillary 23, and is held in a columnar form in the example of FIG. 4. It is preferable there are no air bubbles in the plugs and between the plugs.

The first oil plug 44, the second oil plug 46, and the third oil plug 48 have functions of preventing mixture of the solution plugs on both sides with each other. These oil plugs include e.g. 2CS silicone oil, and the cleaning solution plug 45 includes e.g. 8M guanidine hydrochloride, 0.7% Triton X-100. Note that, in order to suppress movement of the chaotropic agent to the PCR container 30, it is desirable that the cleaning solution plug 45 contains no chaotropic agent.

The reaction solution plug 47 contains a sample necessary for amplification reaction of a target nucleic acid. The sample includes an eluate, DNA polymerase, primer, dNTP (deoxyribonucleotide triphosphate), and buffer.

The eluate is a liquid that releases and elutes the nucleic acid adsorbed to the magnetic beads 7 from the magnetic beads 7 into the liquid. The nucleic acid released from the magnetic beads 7 and eluted into the liquid is a template nucleic acid, and a nucleic acid segment to be amplified in the template nucleic acid is the target nucleic acid. The target nucleic acid includes a DNA (deoxyribonucleic acid) segment, cDNA (complementary DNA) segment, or PNA (peptide nucleic acid). Note that a technique of moving the magnetic beads 7 housed in the tank 3 to the reaction solution plug 47 will be described later.

When the nucleic acid released from the magnetic beads 7 is RNA (ribonucleic acid), to obtain cDNA of the RNA, the plug contains reverse transcriptase, primer for reverse transcriptase, etc. as samples necessary for amplification reaction of the target nucleic acid. Further, when the amplification of the target nucleic acid is quantified using the real-time PCR, the plug contains a probe to which a fluorescent dye is bound such as a TaqMan probe, Molecular Beacon, or cycling probe and a fluorescent dye for intercalator such as SYBR green as samples necessary for amplification reaction of the target nucleic acid.

On the outer wall surface of the tube 20, fixing hooks 25 and a guide plate 26 for attaching the cartridge 1 to a predetermined part of the nucleic acid amplification apparatus are formed.

<<Container>>

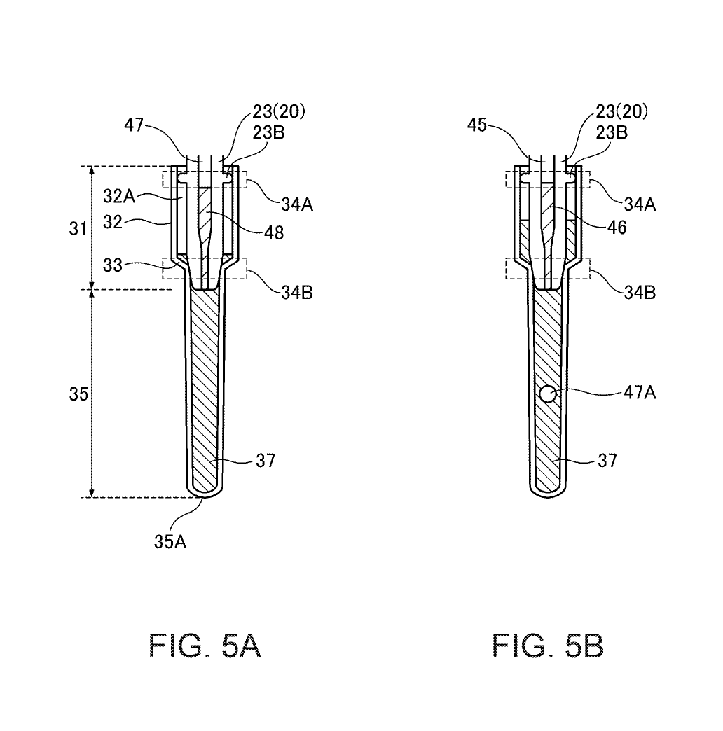

FIG. 5 shows the cartridge with a focus on the PCR container therein. Specifically, FIG. 5(A) shows a state of the PCR container before the plunger 10 is pushed from outside, and FIG. 5(B) shows a state of the PCR container after the plunger 10 is pushed from outside.

The PCR container 30 is a container housing for a droplet 47A pushed out from the tube 20, and has a seal forming part 31 and a channel forming part 35. The droplet 47A is obtained when the reaction solution plug 47 within the capillary 23 is pushed out from the tube 20. Accordingly, the composition of the droplet 47A is the same as the composition of the reaction solution plug 47. That is, the droplet 47A contains the template nucleic acid released from the magnetic beads 7 and the sample necessary for amplification reaction of the target nucleic acid in the template nucleic acid.

The seal forming part 31 is a part into which the tube 20 is inserted and has an oil receiving portion 32 and a step portion 33. The oil receiving portion 32 is a tubular part and functions as a reservoir that receives an oil 37 to fill the channel forming part 35 located on the downstream side of the oil receiving portion 32. A gap exists between the inner wall of the oil receiving portion 32 and the outer wall of the capillary 23 of the tube 20, and the gap serves as an oil receiving space 32A that receives the oil 37 overflowing from the channel forming part 35. The volume of the oil receiving space 32A is larger than the volume of the seal 12A of the plunger 10 sliding in the lower syringe 22 of the tube 20.

The inner wall of the oil receiving portion 32 on the upstream side comes into contact with the annular convex portion of the tube 20, and thereby, an upper seal portion 34A is formed. The upper seal portion 34A is a seal that suppresses leakage of the oil 37 of the oil receiving space 32A to the outside while permitting the passage of the air. In the upper seal portion 34A, a vent hole is formed without leakage of the oil due to the surface tension of the oil. The vent hole of the upper seal portion 34A may be a clearance between the convex portion of the tube 20 and the inner wall of the oil receiving portion 32 or a hole, groove, or cutout formed in the convex portion of the tube 20. Alternatively, the upper seal portion 34A may be formed using an oil absorbing material that absorbs the oil.

The step portion 33 is a part having a step provided on the downstream side of the oil receiving portion 32. The inner diameter of the downstream part of the step portion 33 is smaller than the inner diameter of the oil receiving portion 32. The inner wall of the step portion 33 is in contact with the outer wall of the capillary 23 of the tube 20 on the downstream side. The inner wall of the step portion 33 and the outer wall of the tube 20 come into contact, and thereby, a lower seal portion 34B is formed. The lower seal portion 34B is a seal allowing the oil of the channel forming part 35 to flow to the oil receiving space 32A while resisting the flow. The pressure in the channel forming part 35 is higher than the outside pressure due to pressure loss in the lower seal portion 34B. Even when the oil 37 of the channel forming part 35 is heated, air bubbles are hard to be produced in the oil 37.

The channel forming part 35 is a tubular part serving as a channel in which the droplet 47A moves. The channel forming part 35 is filled with the oil 37. The upstream side of the channel forming part 35 is closed by the terminal end of the tube 20, and the terminal end of the tube 20 opens toward the channel forming part 35. The inner diameter of the channel forming part 35 is larger than the inner diameter of the capillary 23 of the tube 20 and larger than the outer diameter of the droplet 47A. It is desirable that the inner wall of the channel forming part 35 has water-repellency such that the droplet 47A may not be attached.

As described above, in the initial state in which the plunger 10 is not pushed, the rod-like portion 12 of the plunger 10 is located within the upper syringe 21 of the tube 20 (see FIG. 4(A)). Accordingly, the liquid within the tube 20 is not pushed out into the PCR container 30. Note that, in the initial state, the interface of the oil 37 is located on the relatively downstream side of the oil receiving space 32A (see FIG. 5(A)).

On the other hand, when the plunger 10 is pushed, the rod-like portion 12 of the plunger 10 slides within the lower syringe 22 of the tube 20 (see FIG. 4(B)), and thereby, the liquid within the tube 20 is pushed out into the PCR container 30.

Specifically, first, the third oil plug 48 of the tube 20 flows into the channel forming part 35, the inflow oil flows from the channel forming part 35 into the oil receiving space 32A, and the oil interface of the oil receiving space 32A rises. In this regard, the pressure of the liquid in the channel forming part 35 is higher due to pressure loss in the lower seal portion 34B. The third oil plug 48 is pushed out from the tube 20, and then, the reaction solution plug 47 flows from the tube 20 into the channel forming part 35. The inner diameter of the channel forming part 35 is larger than the inner diameter of the capillary 23, and thereby, the reaction solution plug 47 in the columnar shape within the tube 20 becomes the droplet 47A in the oil of the channel forming part 35 (see FIG. 5 (B)). The droplet 47A has larger specific gravity than the oil 37 and sinks in the channel forming part 35.

Nucleic Acid Amplification Apparatus

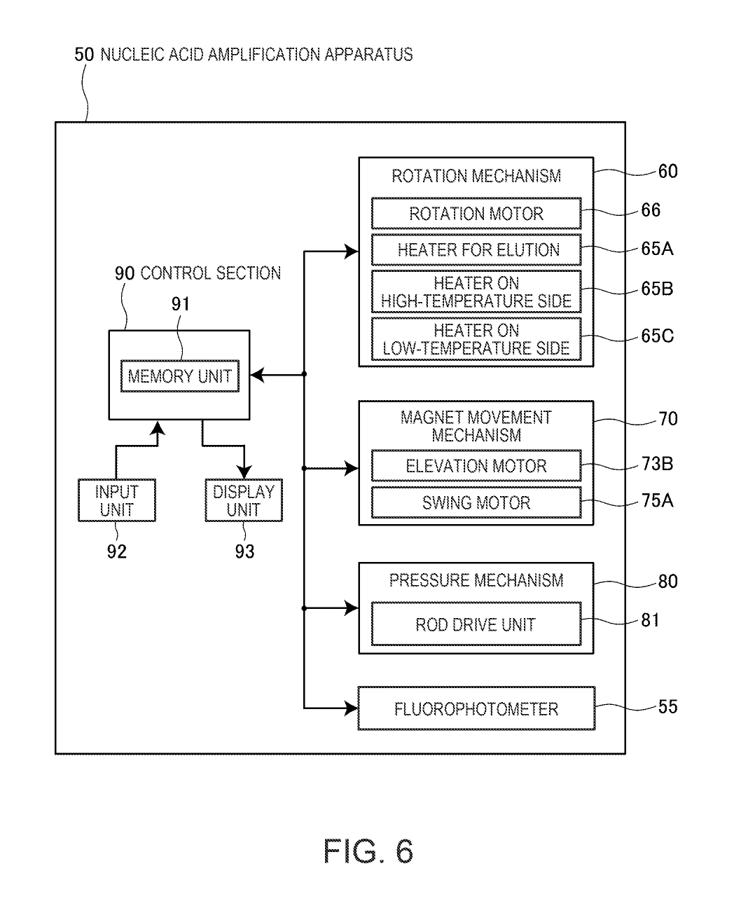

FIG. 6 is a block diagram of a nucleic acid amplification apparatus. As shown in FIG. 6, a nucleic acid amplification apparatus 50 has a rotation mechanism 60, a magnet movement mechanism 70, a pressure mechanism 80, a fluorophotometer 55 and a control section 90.

<Rotation Mechanism>

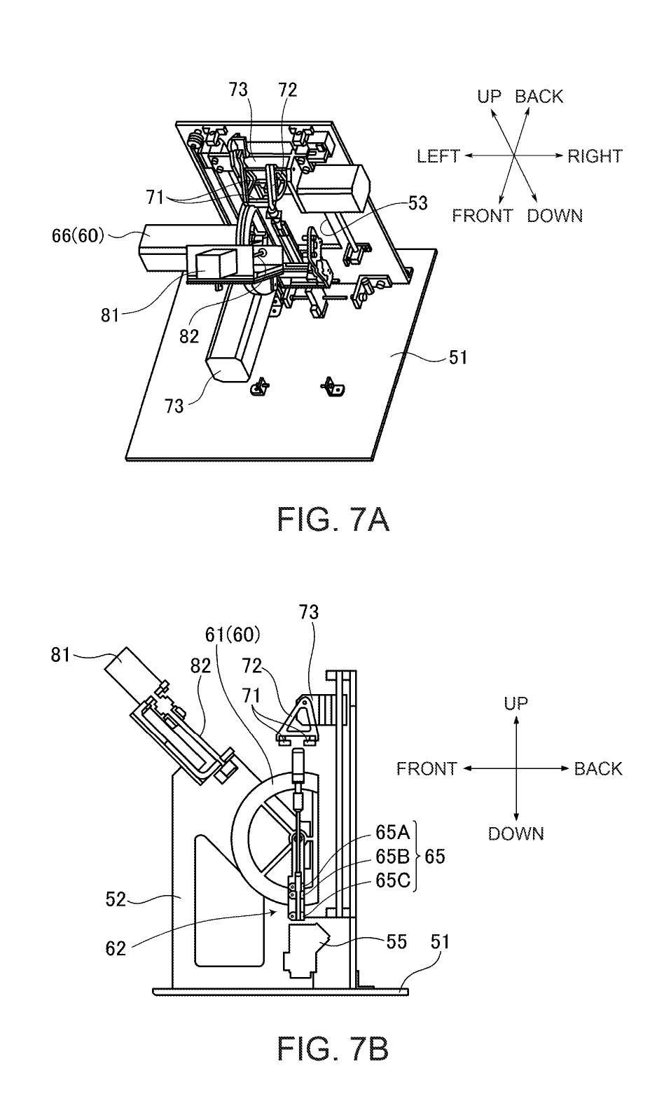

FIG. 7 shows states of the rotation mechanism. FIG. 7(A) is a perspective view of the internal configuration of the nucleic acid amplification apparatus 50, and FIG. 7(B) is a side view of the main configuration of the nucleic acid amplification apparatus 50. In the following explanation of the nucleic acid amplification apparatus 50, as shown in the drawing, up, down, front, back, left, and right are defined. That is, vertical directions when a base 51 of the nucleic acid amplification apparatus 50 is horizontally installed are referred to as “upward and downward directions” and “up” and “down” are defined according to the gravity direction. Further, axis directions of the rotation shaft of the cartridge 1 are referred to as “leftward and rightward directions”, and directions perpendicular to the upward and downward directions and the leftward and right ward directions are referred to as “frontward and backward directions”. The side of a cartridge insertion opening 53 as seen from the rotation shaft of the cartridge 1 is referred to as “back” and the opposite side is referred to as “front”. The right side of the leftward and right ward directions as seen from the front side is referred to as “right” and the left side is referred to as “left”.

As shown in FIG. 7, the rotation mechanism 60 has a rotator 61 and a rotation motor 66. In the rotator 61, an attachment part 62 to which the cartridge 1 is attached and a heater 65 are provided. The rotator 61 rotates about a rotation shaft supported by a support 52 fixed to the base 51 without changing the relative positions of the cartridge 1 and the heater 65.

The rotation motor 66 is a power source that rotates the rotator 61, and rotates the rotator 61 so that the cartridge 1 may be vertically inverted according to an instruction from the control section 90.

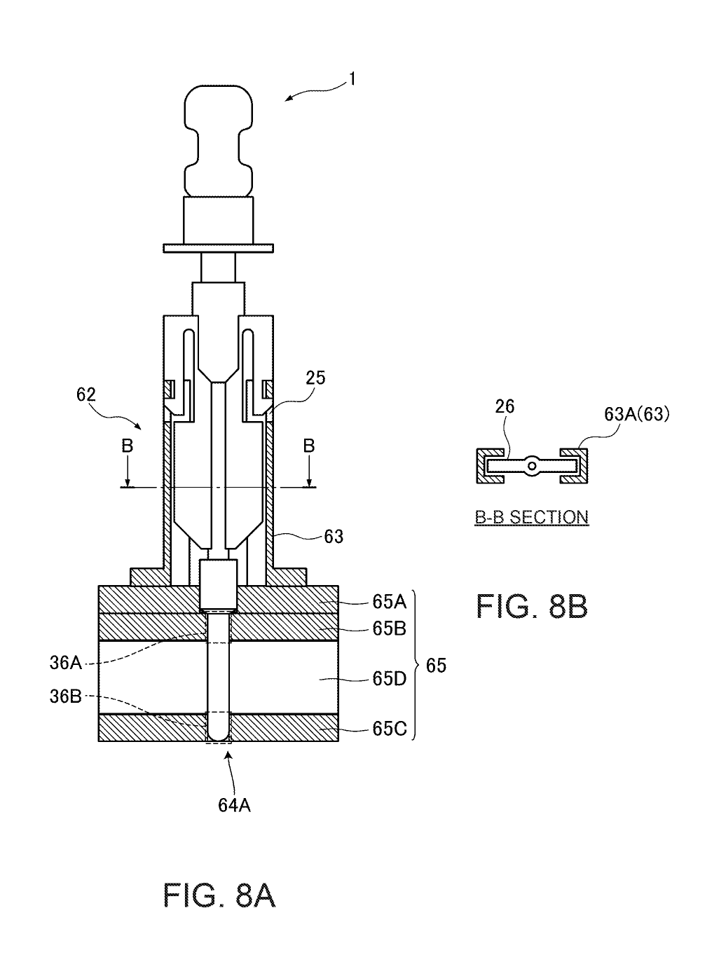

FIG. 8 shows a state in which the cartridge has been attached to the attachment part. As shown in FIG. 8, the attachment part 62 has fixing portions 63 that fix the tube 20 of the cartridge 1 and an insertion hole 64A that fixes the PCR container 30.

The insertion hole 64A is formed in the heater 65. The heater 65 of the embodiment has a heater for elution 65A for heating to a temperature at which the release reaction of the nucleic acid from the magnetic beads 7 progresses, a heater on high-temperature side 65B for heating to a temperature at which denaturation reaction of the target nucleic acid progresses, and a heater on low-temperature side 65C for heating to a temperature at which synthesis reaction (annealing reaction and elongation reaction) of the target nucleic acid progresses. The insertion hole 64A penetrates through these heater for elution 65A, heater on high-temperature side 65B, and heater on low-temperature side 65C.

The fixing portions 63 are members provided on the sides of the opening of the insertion hole 64A to face each other. In the fixing portions 63, guide rails 63A that guide the cartridge 1 to the insertion hole 64A while constraining the guide plate 26 of the cartridge 1 in the forward and backward directions are provided.

In the attachment part 62, when the PCR container 30 of the cartridge 1 guided by the guide rails 63A is inserted into the insertion hole 64A, the fixing hooks 25 of the cartridge 1 are caught in the notch parts of the fixing portions 63, and thereby, the cartridge 1 is attached. Here, a part of the heater also serves as the attachment part 62, however, the attachment part 62 and the heater may separate. Further, the attachment part 62 is fixed indirectly to the rotator 61 via the heater for elution 65A, however, may be directly provided on the rotator 61. Furthermore, the number of cartridges 1 that can be attached to the attachment part 62 is not limited to one, but may be more than one.

When the cartridge 1 is attached to the attachment part 62, the reaction solution plug 47 of the cartridge 1 is surrounded by the heater for elution 65A. The heater for elution 65A heats the reaction solution plug 47 to e.g. 50° C. Thereby, the release of the nucleic acid from the magnetic beads 7 that have been moved from the tank 3 to the reaction solution plug 47 is promoted.

Further, when the cartridge 1 is attached to the attachment part 62, a first region 36A on one side of the channel forming part 35 in the PCR container 30 of the cartridge 1 is surrounded by the heater on high-temperature side 65B. The heater on high-temperature side 65B heats the first region 36A to e.g. 95 to 100° C.

Furthermore, when the cartridge 1 is attached to the attachment part 62, a second region 36B on the other side of the channel forming part 35 in the PCR container 30 of the cartridge 1 is surrounded by the heater on low-temperature side 65C. The heater on low-temperature side 65C heats the second region 36B to e.g. 50 to 75° C.

In this manner, the first region 36A of the PCR container 30 is heated to the temperature at which the denaturation reaction of the target nucleic acid progresses and the second region 36B of the PCR container 30 is heated to the temperature at which the synthesis reaction of the target nucleic acid progresses, and thereby, a temperature gradient is formed in the oil 37 filling the channel forming part 35.

Note that a spacer 65D that suppresses thermal conduction between the heater on high-temperature side 65B and the heater on low-temperature side 65C is provided between the heater on high-temperature side 65B and the heater on low-temperature side 65C. In the spacer 65D, a through hole is formed in a position along the longitudinal direction of the insertion hole 64A of the heater on high-temperature side 65B and the heater on low-temperature side 65C, and hindrance of the insertion of the PCR container 30 of the cartridge 1 into the insertion hole 64A is prevented.

<Magnet Movement Mechanism>

As shown in FIG. 7(B), the magnet movement mechanism 70 has a pair of magnets 71, an arm 72 that holds the pair of magnets 71, and an elevating unit 73 that moves up and down the arm 72, and is driven according to an instruction from the control section 90.

That is, the magnet movement mechanism 70 attracts the magnetic beads 7 within the tank 3 of the cartridge 1 attached to the attachment part 62 toward the magnets 71, and moves the magnetic beads 7 to the reaction solution plug 47 along the cartridge main body 9.

When the nucleic acid adsorbed to the magnetic beads 7 is released from the magnetic beads 7 in the reaction solution plug 47, the magnet movement mechanism 70 returns the magnetic beads 7 within the reaction solution plug 47 to the tank 3 along the cartridge main body 9.

<Pressure Mechanism>

As shown in FIG. 7(B), the pressure mechanism 80 has a rod drive unit 81 and a rod 82 that pushes the mount 11A of the plunger 10 in the cartridge 1. The direction in which the rod 82 pushes the mount 11A is not the upward and downward directions, but inclined at 45 degrees with respect to the upward and downward directions. Accordingly, when the plunger 10 is pushed by the pressure mechanism 80, the rotator 61 is rotated to by 45 degrees so that the longitudinal direction of the cartridge 1 may be the same as the movement direction of the rod 82.

In this state, the rod drive unit 81 pushes the rod 82 to the mount 11A along the longitudinal direction of the cartridge 1 according to an instruction from the control section 90. Thereby, the reaction solution plug 47 of the cartridge 1 is housed as the droplet 47A in the PCR container 30.

Note that the direction in which the plunger 10 is pushed by the rod 82 is inclined at 45 degrees with respect to the upward and downward directions, and thereby, the placement of the pressure mechanism 80 not to interfere with the elevating unit 73 is easier. Further, the direction in which the plunger 10 is pushed by the rod 82 is inclined at 45 degrees with respect to the upward and downward directions, and thereby, the dimension of the nucleic acid amplification apparatus 50 in the upward and downward directions may be made smaller.

<Fluorophotometer>

The fluorophotometer 55 is a measuring instrument that measures the fluorescence intensity of the droplet 47A housed in the PCR container 30, and placed to face the terminal end of the cartridge 1 to be attached to the attachment part 62 at a predetermined distance as shown in FIG. 7(B).

The fluorophotometer 55 radiates excitation light corresponding to the fluorescent dye contained in the droplet 47A according to a measurement instruction from the control section 90, and measures the fluorescence intensity emitted in the droplet 47A. Further, the fluorophotometer 55 provides data showing the fluorescence intensity obtained as a measurement result to the control section 90. Note that the fluorophotometer 55 may measure the fluorescence intensity corresponding to one fluorescent dye or measure the fluorescence intensity corresponding to a plurality of fluorescent dyes.

<Control Section>

As shown in FIG. 6, the control section 90 has a memory unit 91, and an input unit 92 and a display unit 93 are connected to the control section 90. The memory unit 91 contains an area in which programs are stored, an area in which various kinds of data including setting data input from the input unit 92 and data obtained by nucleic acid amplification processing are stored, and an area in which the programs and the data are developed.

The control section 90 appropriately controls the rotation mechanism 60, the magnet movement mechanism 70, the pressure mechanism 80, and the fluorophotometer 55 based on the programs and setting data stored in the memory unit 91, and appropriately executes nucleic acid elution processing, droplet formation processing, thermal cycling processing or amplification analysis processing.

<<Nucleic Acid Elution Processing>>

For example, the nucleic acid elution processing is executed after the control section 90 senses the attachment of the cartridge 1 based on a sensor (not shown) provided in a predetermined part of the attachment part 62.

That is, the control section 90 controls the elevating unit 73 to place the pair of magnets 71 located in the retracted position at a predetermined distance apart from the tank 3 of the cartridge 1 attached to the attachment part 62 around the outer side surface of the tank 3. Thereby, the magnetic beads 7 within the tank 3 are attracted by the magnets 71.

Then, the control section 90 moves down the elevating unit 73 at a velocity at which the magnetic beads 7 may follow the movement of the magnets 71 in a predetermined period, and places the pair of magnets 71 around the outer side surface of the tube 20 in which the reaction solution plug 47 is placed. Thereby, the magnetic beads 7 are introduced into the reaction solution plug 47.

Further, the control section 90 drives the heater for elution 65A in a predetermined period from the time before the magnetic beads 7 are introduced into the reaction solution plug 47, and heats the reaction solution plug 47 to e.g. 50° C. Thereby, the nucleic acid adsorbed to the magnetic beads 7 are released from the magnetic beads 7. When the nucleic acid is RNA, reverse transcription reaction progresses after the release from the magnetic beads 7, and cRNA is obtained.

After a lapse of a period set so that the magnetic beads 7 should be retained in the reaction solution plug 47, the control section 90 moves up the elevating unit 73 in a predetermined period at a velocity at which the magnetic beads 7 may follow the movement of the magnets 71, and returns the magnetic beads 7 to the tank 3.

Then, the control section 90 switches to a velocity at which the magnetic beads 7 are hard to follow the movement of the magnets 71, and returns the magnets 71 to the above described retracted position. Thereby, the magnetic beads 7 returned to the tank 3 separate from the magnets 71 and stay within the tank 3. Here, the nucleic acid elution processing ends.

Note that, if the magnetic beads 7 have been moved to the upper syringe 21, the magnetic beads 7 are not introduced into the PCR container 30 when the plunger 10 is pushed. Therefore, the control section 90 changes the velocity to the velocity at which the magnetic beads 7 are hard to follow the movement of the magnets 71 from the time when the magnets 71 are placed around the outer side surface of the tube 20 in which the upper syringe 21 is placed so that the magnetic beads 7 may separate from the magnets 71 in the upper syringe 21.

<<Droplet Formation Processing>>

The droplet formation processing is executed after the execution of the above described nucleic acid elution processing. That is, the control section 90 rotates the rotator 61 from the reference position by 45 degrees so that the longitudinal direction of the cartridge 1 may be made the same as the movement direction of the rod 82.

Then, the control section 90 drives the rod drive unit 81 and moves the rod 82 from the reference position at a predetermined velocity in a predetermined period to push the plunger 10 until the mount 11A of the plunger 10 comes into contact with the upper edge of the tube 20. Thereby, the seal 12A of the rod-like portion 12 in the plunger 10 is fitted in the lower syringe 22 of the tube 20, and then, slides (see FIG. 2(B)), and the droplet 47A corresponding to the volume of sliding of the seal 12A within the lower syringe 22 is pushed out into the channel forming part 35 of the PCR container 30 (see FIG. 5(B)).

Then, the control section 90 returns the rod 82 and the rotator 61 to the original reference positions. Here, the droplet formation processing ends.

<<Thermal Cycling Processing>>

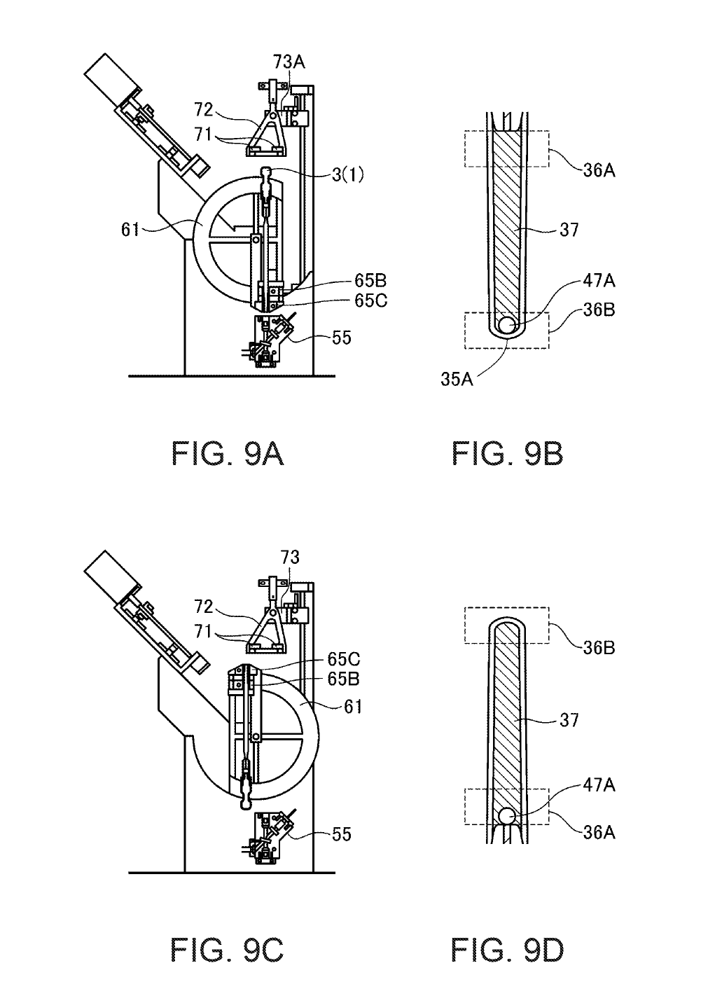

FIG. 9 shows states of thermal cycling processing. Specifically, FIGS. 9(A) and 9(B) show states of the denaturation stage of the target nucleic acid and FIGS. 9(C) and 9(D) show states of the synthesis stage of the target nucleic acid.

The thermal cycling processing is executed after the above described droplet formation processing is executed. That is, the control section 90 drives the heater on high-temperature side 65B provided on the rotator 61 and heats the first region 36A of the PCR container 30 to the temperature at which the denaturation reaction of the target nucleic acid progresses. Further, the control section 90 drives the heater on low-temperature side 65C provided on the rotator 61 and heats the second region 36B of the PCR container 30 to the temperature at which the synthesis reaction of the target nucleic acid progresses. Thereby, a temperature gradient is formed in the oil 37 within the channel forming part 35 of the PCR container 30.

A predetermined period is necessary after the heater on high-temperature side 65B and the heater on low-temperature side 65C are driven until the oil 37 within the channel forming part 35 in the first region 36A reaches e.g. 95° C. and the oil 37 within the channel forming part 35 in the second region 36B reaches e.g. 60° C. The amplification reaction of the target nucleic acid does not properly progress in this period, and the control section 90 waits in the period as a waiting period.

In this regard, as shown in FIG. 9(A), the rotator 61 is located in the reference position in which the tank side of the cartridge 1 attached to the attachment part 62 is placed on the upside and the PCR container side of the cartridge 1 is placed on the downside. When the rotator 61 is located in the reference position, as shown in FIG. 9(B), the droplet 47A sinks under the weight of its own and stays in the second region 36B. Therefore, the target nucleic acid contained in the droplet 47A does not move to the first denaturation stage.

When the above described waiting period elapses, the control section 90 rotates the rotator 61 by 180 degrees. In this case, as shown in FIG. 9(C), the rotator 61 is located in the inverted position in which the tank side of the cartridge 1 attached to the attachment part 62 is placed on the downside and the PCR container side of the cartridge 1 is placed on the upside. When the rotator 61 is located in the inverted position, as shown in FIG. 9(D), the droplet 47A sinks under the weight of its own and moves to the first region 36A. Therefore, the target nucleic acid contained in the droplet 47A moves to the denaturation stage.

Further, the control section 90 stops the rotator 61 from the time when finishing rotation of the rotator 61 by 180 degrees (stopping the rotator 61) in a denaturation reaction period set as a period at the denaturation stage necessary for the denaturation reaction of the target nucleic acid. Thereby, the denaturation reaction of the target nucleic acid contained in the droplet 47A progresses. Note that the denaturation reaction period is set to at least a longer period than the period in which the droplet 47A moves from one end to the other end of the channel forming part 35 by the rotation of the rotator 61.

Then, when denaturation reaction period elapses, the control section 90 rotates the rotator 61 by 180 degrees and switches the rotator 61 from the inverted position to the reference position to move the droplet 47A to the second region 36B. Thereby, the target nucleic acid contained in the droplet 47A moves to the synthesis stage.

Further, the control section 90 stops the rotator 61 from the time when finishing rotation of the rotator 61 by 180 degrees (stopping the rotator 61) in a synthesis reaction period set as a period at the synthesis stage necessary for the synthesis reaction of the target nucleic acid. Thereby, the annealing reaction and the elongation reaction of the target nucleic acid contained in the droplet 47A progress. Note that the synthesis reaction period is set to at least a longer period than the period in which the droplet 47A moves from one end to the other end of the channel forming part 35 like the above described denaturation period.

In this manner, the control section 90 alternately switches between the above described inverted position and reference position and repeats a cycle through the denaturation stage at which the droplet 47A is moved to and retained in the first region 36A and the synthesis stage at which the droplet 47A is moved to and retained in the second region 36B at a plurality of times. The number of cycles to be repeated is set in the control section 90, to e.g. 30 times.

Now, in the embodiment, the periods of the respective cycles following the time as a specified-numbered time counted from the first time are made shorter than the periods of the respective cycles until the time as the specified-numbered time counted from the first time. The specified number of times is set in the control section 90 within a range e.g. from one to fifteen times. Note that it is desirable that the ratio of the number of cycles to be shortened to the number of cycles to be repeated is less than 50%.

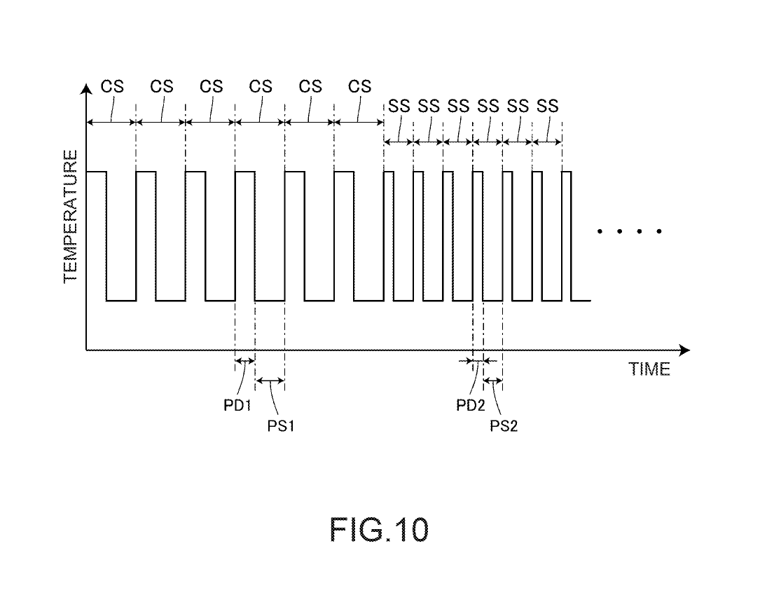

FIG. 10 schematically shows temperature shifts of the droplet. Note that, in FIG. 10, for convenience, the movement period of the droplet 47A between the denaturation reaction period and the synthesis reaction period and the movement period of the droplet 47A between the synthesis reaction period and the denaturation reaction period are omitted.

As shown in FIG. 10, in the embodiment, a reference cycle CS as a reference cycle and a shortened cycle SS as a shortened cycle than the reference cycle CS are set in the control section 90. The reference cycle CS includes a first denaturation reaction period PD1 as a reference denaturation reaction period and a first synthesis reaction period PS1 as a reference synthesis reaction period. For example, the first denaturation reaction period PD1 is set within a range from 5 seconds to 60 seconds and the first synthesis reaction period PS1 is set within a range from 6 seconds to 60 seconds. The shortened cycle SS includes a second denaturation reaction period PD2 shorter than the first denaturation reaction period PD1 and a second synthesis reaction period PS2 shorter than the first synthesis reaction period PS1. For example, the second denaturation reaction period PD2 is set within a range from 2 seconds to 5 seconds and the second synthesis reaction period PS2 is set within a range from 4 seconds to 6 seconds.

As described above, the control section 90 repeats the reference cycle CS until the time as the specified-numbered time counted from the first time and repeats the shortened cycle SS from the time following the time to the last time. <<Amplification Analysis Processing>>

The amplification analysis processing is executed in parallel to the thermal cycling processing in the same period. That is, the control section 90 gives a measurement instruction in each synthesis reaction period (first synthesis reaction period PS1 and second synthesis reaction period PS2) to the fluorophotometer 55, and stores data showing the fluorescence intensity provided from the fluorophotometer 55 as the measurement instruction result in the memory unit 91.

Note that, as shown in FIG. 9(A) and 9(B), in the synthesis reaction period, the rotator 61 is located in the reference position, and the droplet 47A within the PCR container 30 sinks to a bottom 35A of the PCR container 30. However, immediately after located in the reference position, the droplet 47A may not reach the bottom 35A of the PCR container 30. Therefore, it is desirable that the period in which the control section 90 gives the measurement instruction to the fluorophotometer 55 is after a predetermined time lapses from the time when the rotation of the rotator 61 from the inverted position to the reference position is finished. Particularly, it is desirable that the period is immediately before the rotation from the reference position to the inverted position.

Further, the control section 90 reads data showing the fluorescence intensity for the number of times set as the number of cycles to be repeated according to a command input from the input unit 92 from the memory unit 91, and generates an amplification curve showing the shifts of the fluorescence intensity corresponding to the number of cycles based on the data. Then, when generating the amplification curve, the control section 90 determines good or bad with respect to reference amplification efficiency based on the amplification curve, and appropriately allows the display unit 93 to display both or either of the determination result and the amplification curve.

<Thermal Cycling Processing Procedure>

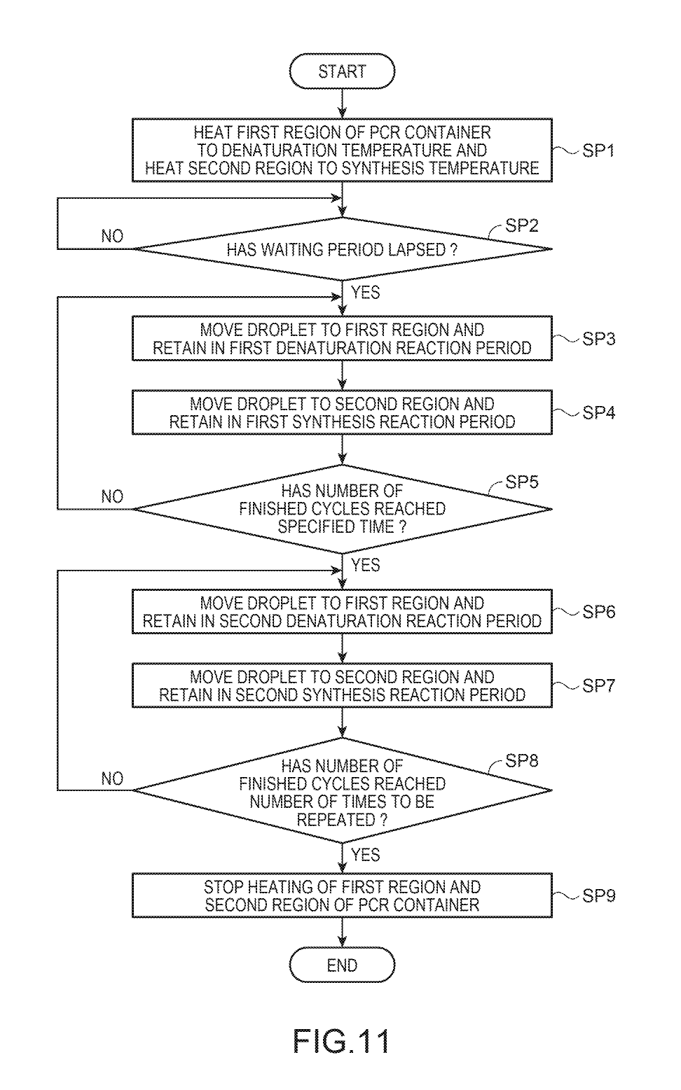

FIG. 11 is a flowchart showing a thermal cycling processing procedure of the control section. As shown in FIG. 11, the control section 90 moves to step SP1 after the execution of the nucleic acid elution processing, and heats the first region 36A in the PCR container 30 to a denaturation temperature set as a temperature at which the denaturation reaction of the target nucleic acid progresses. Further, the control section 90 heats the second region 36B in the PCR container 30 to a synthesis temperature set as a temperature at which the synthesis reaction of the target nucleic acid progresses, and moves to step SP2.

At step SP2, the control section 90 waits until awaiting period set such that an object to be heated reaches a target temperature from the start of heating lapses, and, if the waiting period has lapsed, moves to step SP3.

At step SP3, the control section 90 moves the droplet 47A to the denaturation temperature region (first region 36A) of the PCR container 30 by rotating the rotator 61 from the reference position to the inverted position. Then, the control section 90 continues to stop the rotator 61 and retains the droplet 47A in the denaturation temperature region of the PCR container 30 until the first denaturation reaction period PD1 lapses from the time when the rotator 61 is located in the inverted position. If the first denaturation reaction period PD1 has lapsed, the control section 90 moves to step SP4.

At step SP4, the control section 90 moves the droplet 47A to the synthesis temperature region (second region 36B) of the PCR container 30 by rotating the rotator 61 from the inverted position to the reference position. Then, the control section 90 continues to stop the rotator 61 and retains the droplet 47A in the synthesis temperature region of the PCR container 30 until the first synthesis reaction period PS1 lapses from the time when the rotator 61 is located in the reference position. If the first synthesis reaction period PS1 has lapsed, the control section 90 moves to step SP5.

At step SP5, the control section 90 recognizes whether or not the number of finished cycles has reached a specified number of times set as the number of times at which the cycle period should be switched. Here, if the number of finished cycles has not reached the specified number of times, the control section 90 increases the number of finished cycles by one, and then, returns to step SP3 and repeats the above described processing. On the other hand, if the number of finished cycles has reached the specified number of times, the control section 90 increases the number of finished cycles by one, and then, moves to step SP6.

At step SP6, the control section 90 moves the droplet 47A to the denaturation temperature region (first region 36A) of the PCR container 30 by rotating the rotator 61 from the reference position to the inverted position. Then, the control section 90 continues to stop the rotator 61 and retains the droplet 47A in the denaturation temperature region of the PCR container 30 until the second denaturation reaction period PD2 lapses from the time when the rotator 61 is located in the inverted position. If the second denaturation reaction period PD2 has lapsed, the control section 90 moves to step SP7.

At step SP7, the control section 90 moves the droplet 47A to the synthesis temperature region (second region 36B) of the PCR container 30 by rotating the rotator 61 from the inverted position to the reference position. Then, the control section 90 continues to stop the rotator 61 and retains the droplet 47A in the synthesis temperature region of the PCR container 30 until the second synthesis reaction period PS2 lapses from the time when the rotator 61 is located in the reference position. If the second synthesis reaction period PS2 has lapsed, the control section 90 moves to step SP8.

At step SP8, the control section 90 recognizes whether or not the number of finished cycles has reached the number of times set as the number of cycles to be repeated. Here, if the number of finished cycles has not reached the number of cycles to be repeated, the control section 90 increases the number of finished cycles by one, and then, returns to step SP6 and repeats the above described processing. On the other hand, if the number of finished cycles has reached the number of cycles to be repeated, the control section 90 moves to step SP9.

At step SP9, the control section 90 stops heating of the first region 36A and the second region 36B in the PCR container 30, and then, ends the thermal cycling processing.

<Brief>

As described above, in the embodiment, the first region 36A of the PCR container 30 is heated to the denaturation temperature of the target nucleic acid, and the second region 36B independent of the first region 36A is heated to the synthesis temperature of the target nucleic acid.

Further, the cycle through the denaturation stage at which the droplet 47A within the PCR container 30 is moved to and retained in the denaturation temperature region (first region 36A) and the synthesis stage at which the droplet 47A is moved to and retained in the synthesis temperature region (second region 36B) is repeated at the plurality of times.

Here, the periods of the respective cycles following the time as the specified-numbered time counted from the first time is made shorter than the periods of the respective cycles until the time as the specified-numbered time counted from the first time. That is, of the plurality of cycles, the first several cycles are set to the reference cycles CS and the respective cycles after the first several cycles are set to the shortened cycles SS. Accordingly, compared to the case where all of the plurality of cycles are set to the reference cycles CS, the periods of the respective cycles after the first several cycles are shortened.

In the PCR method, factors that make the amplification reaction difficult include the case where the chain length of the nucleic acid collected as a template is longer and denaturation is harder to occur, the case where a supercoiled part exists in the nucleic acid and denaturation is harder to occur, the case where the elongated chain is not completely elongated in the synthesis reaction of the nucleic acid and the reaction ends. In the cases, the amplification cycles of others than the target nucleic acid progress and the amplification efficiency of the PCR product as a target is significantly reduced.

In the embodiment, the first several cycles of the plurality of times of cycles are set to the reference cycles CS and the respective cycles after the several cycles are set to the shortened cycles SS. Accordingly, even when the factor that makes the amplification reaction difficult exists in the target nucleic acid to be amplified, compared to the case where the first several cycles are not secured to be longer than the subsequent cycles, a PCR product having a short linear chain length and easily denaturated and a PCR product having a full length may be easily obtained and the rate is larger. Therefore, even when the respective cycles after the first several cycles are set to be shorter than the periods of the several cycles, at least the minimum amplification efficiency that should be secured is maintained.

As described above, according to the embodiment, the amplification reaction period (cycle time) may be shortened while the certain amplification efficiency is maintained. As a result, shortening of the production time of the PCR product while suppressing the reduction of the amplification efficiency is realized.

Note that, in the case of the embodiment, the cycle through the denaturation stage at which the droplet 47A in the PCR container 30 is moved to and stayed in the denaturation temperature region (first region 36A) and the synthesis stage at which the droplet 47A is moved to and stayed in the synthesis temperature region (second region 36B) is repeated at the plurality of times. Accordingly, compared to the case where the temperature in the PCR container 30 is switched between the denaturation temperature and the synthesis temperature of the target nucleic acid, the waiting period for the temperature is omitted and the amplification reaction period (cycle time) is shortened by the amount.

(2) Second Embodiment

Next, the second embodiment will be explained. Note that the cartridge of the embodiment is the same as that of the above described first embodiment, and the explanation of the cartridge will be omitted. Further, the nucleic acid amplification apparatus is the same as that of the above described first embodiment except thermal cycling processing of the control section 90 and the explanation of the others than the thermal cycling processing will be omitted.

<<Thermal Cycling Processing>>

The thermal cycling processing of the embodiment is the same with the thermal cycling processing of the first embodiment in that periods of part of cycles of the plurality of cycles are made shorter than the periods of the other cycles. On the other hand, the temporal parts of the cycles to be shortened of the plurality of cycles are different between the thermal cycling processing of the embodiment and the thermal cycling processing of the first embodiment.

That is, in the first embodiment, the periods of the respective cycles following the time as the specified-numbered time counted from the first time are made shorter than the periods of the respective cycles to the time as the specified-numbered time counted from the first time.

On the other hand, in the embodiment, the periods of the respective cycles to the time as a specified-numbered time counted from the first time are made shorter than the periods of the respective cycles of the times following the time.

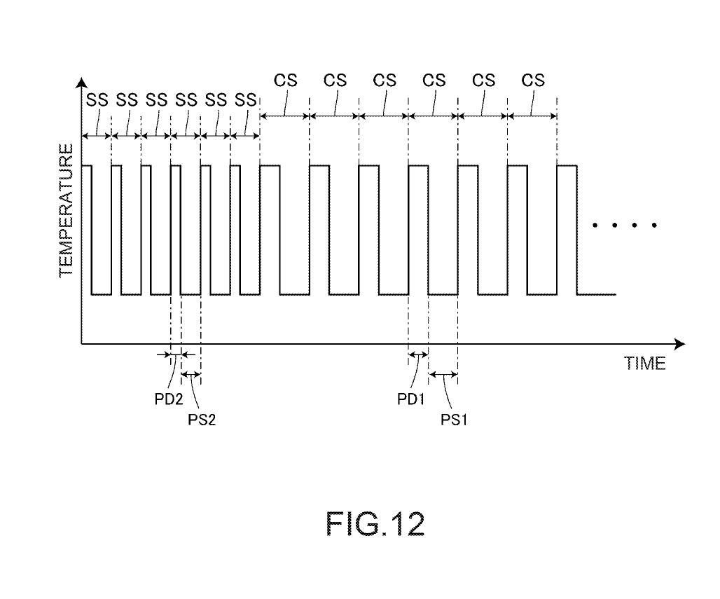

FIG. 12 schematically shows temperature shifts of the droplet in the second embodiment. Note that, in FIG. 12, as is the case shown in FIG. 10, the movement period of the droplet 47A between the denaturation reaction period and the synthesis reaction period and the movement period of the droplet 47A between the synthesis reaction period and the denaturation reaction period are omitted.

As shown in FIG. 12, the control section 90 of the embodiment repeats the shortened cycle SS until the time as the specified-numbered time counted from the first time and repeats the reference cycle CS from the time following the time to the last time.

<Thermal Cycling Processing Procedure>

In the thermal cycling processing of the first embodiment (see FIG. 10), the shortened cycles SS are executed after the reference cycles CS, however, in the thermal cycling processing of the embodiment (see FIG. 12), the reference cycles CS are executed after the shortened cycles SS. That is, the sequences of the reference cycles CS and the shortened cycles SS are opposite between the thermal cycling processing of the embodiment and the thermal cycling processing of the first embodiment.

Therefore, the thermal cycling processing procedure of the embodiment is different from the thermal cycling processing procedure of the above described first embodiment (see FIG. 11) only in step SP3, step SP4, step SP6 and step SP7.

Specifically, at step SP3 in the thermal cycling processing procedure of the embodiment, the processing at the above described step SP6 is executed, and, at step SP4, the processing at the above described step SP7 is executed. Further, at step SP6, the processing at the above described step SP3 is executed, and, at step SP7, the processing at the above described step SP4 is executed.

<Brief>

As described above, in the embodiment, the periods of the respective cycles to the time as the specified-numbered time counted from the first time are made shorter than the periods of the respective cycles of the times following the time. That is, the first several cycles of the plurality of times of cycles are set to the shortened cycles SS and the respective cycles after the several cycles are set to the reference cycles CS. Accordingly, compared to the case where all of the plurality of times of cycles are set to the reference cycles CS, the periods of the respective cycles in the first several cycles are shortened.

In the PCR method, factors that make non-specific synthesis reaction easy include the case where the amount of the nucleic acid collected as a template differs depending on the skill of the collector or the case where the nucleic acid of the living organism to be amplified and a nucleic acid separately originating from other than the living organism are mixed according to the collecting method. In the cases, the amplification cycles of others than the target nucleic acid progress and the amplification efficiency of the PCR product as a target is significantly reduced.

In the embodiment, the first several cycles of the plurality of times of cycles are set to the shortened cycles SS and the respective cycles after the several cycles are set to the reference cycles CS. Accordingly, even when the factor that makes the non-specific synthesis reaction easy exists, compared to the case where the first several cycles are also set to the reference cycles CS, the amount of the non-specific synthesis reaction is smaller and the rate of the non-specific PCR products to the target PCR products is smaller. Therefore, even when the non-specific synthesis reaction occurs in the respective cycles after the several cycles, a certain amount of non-specific synthesis reaction or more is secured and at least the minimum amplification efficiency that should be secured is maintained.

As described above, according to the embodiment, the amplification reaction period (cycle time) may be shortened while the certain amplification efficiency is maintained. As a result, shortening of the production time of the PCR product while suppressing the reduction of the amplification efficiency is realized.

(3) Modified Examples

In the above described embodiments, the second denaturation reaction period PD2 in the shortened cycle SS is made shorter than the first denaturation reaction period PD1, and the second synthesis reaction period PS2 in the shortened cycle SS is made shorter than the first synthesis reaction period PS1 (see FIGS. 10 and 12).

However, the denaturation reaction period in part of the shortened cycles SS of the respective shortened cycles SS may not necessarily be shorter than the first denaturation reaction period PD1.

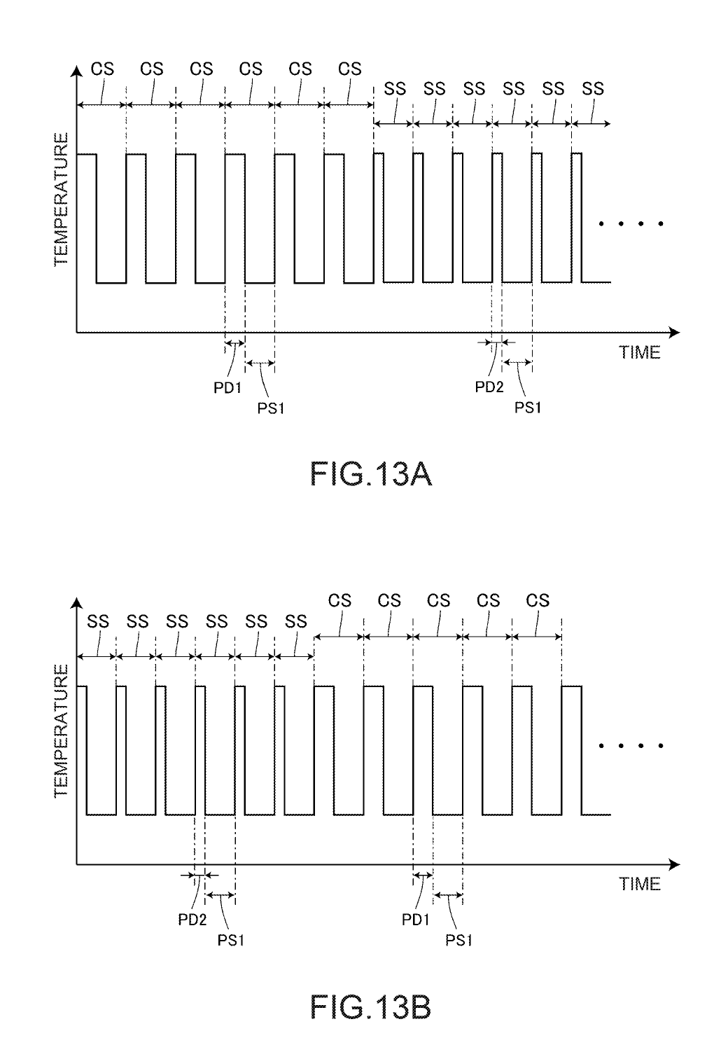

Further, as shown in FIG. 13, the second denaturation reaction period PD2 in the shortened cycle SS may be made shorter than the first denaturation reaction period PD1, and the synthesis reaction period in the shortened cycle SS is not made shorter, but nearly equal to the first synthesis reaction period PS1. Note that FIG. 13(A) corresponds to the case where only the second denaturation reaction period PD2 of the first embodiment is made shorter than the first denaturation reaction period PD1, and FIG. 13(B) corresponds to the case where only the second denaturation reaction period PD2 of the second embodiment is made shorter than the first denaturation reaction period PD1.

In this case, compared to the above described embodiments in which the second synthesis reaction period PS2 is shorter than the first synthesis reaction period PS1, the period of fluorescence measurement executed in the second synthesis reaction period PS2 may be secured to be longer. Therefore, the period of the whole amplification reaction may be shortened without reduction of fluorescence measurement accuracy. Note that, in the example shown in FIG. 13, all denaturation reaction periods in each shortened cycle SS are made shorter than the first denaturation reaction period PD1, however, part of the denaturation reaction periods may not necessarily be made shorter than the first denaturation reaction period PD1.

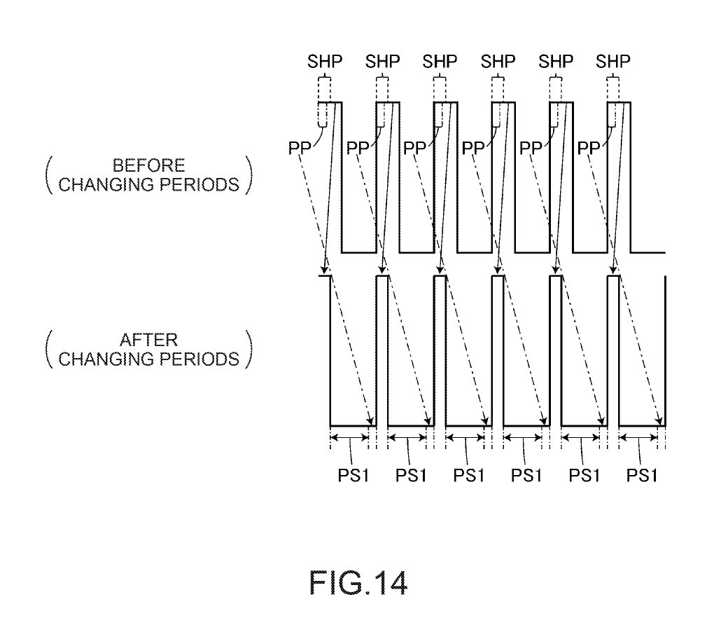

Or, when the second denaturation reaction period PD2 is made shorter than the first denaturation reaction period PD1 and the second synthesis reaction period PS2 is not made shorter than the first synthesis reaction period PS1, as shown in FIG. 14, a partial period PP of a period SHP of the shortened amount in which the second denaturation reaction period PD2 is made shorter than the first denaturation reaction period PD1 may be assigned to the first synthesis reaction period PS1. In this manner, reduction of the amplification efficiency may be suppressed by taking the longer synthesis reaction period that is likely to affect the amplification efficiency while shortening the period of the whole amplification reaction. Note that, in the example shown in FIG. 14, all denaturation reaction periods in each cycle are made shorter than the first denaturation reaction period PD1 and the partial period PP of the amount of shortening of each denaturation reaction period is assigned to the first synthesis reaction period PS1 of the cycle. However, only part of the denaturation reaction periods in each cycle may be made shorter than the first denaturation reaction period PD1 and the partial period PP of the shortened amount may be assigned to the first synthesis reaction period PS1 other than the first synthesis reaction period PS1 of the cycle.

Or, the denaturation reaction period in the shortened cycle SS may not be shortened, but may be nearly equal to the first denaturation reaction period PD1 and the second synthesis reaction period PS2 in the shortened cycle SS may be made shorter than the first synthesis reaction period PS1.

In the above described first embodiment, the cycles after the first several cycles of the plurality of cycles are set to the shortened cycles SS (see FIG. 10) and, in the above described second embodiment, the first several cycles of the plurality of cycles are set to the shortened cycles SS (see FIG. 12). However, the shortened cycle SS and the reference cycle CS may be alternately repeated. Note that the number of the shortened cycles SS and the number of the reference cycles CS to be alternately repeated may be the same or different.

Or, a first cycle pattern in which the first several cycles of the plurality of cycles are set to the shortened cycles SS and a second cycle pattern in which the cycles subsequent to the first several cycles of the plurality of cycles are set to the shortened cycles SS may be switched. The switching method includes e.g. a technique by the control unit 90 switching between the first cycle pattern and the second cycle pattern according to a switching command from the input unit 92.

In conclusion, when the periods of part of cycles of the plurality of cycles are made shorter than the periods of the other cycles, the amplification reaction period may be shortened while the reduction of the amplification efficiency is suppressed as is the case of the above described embodiments.

In the above described embodiments, the start times of the denaturation reaction period and the synthesis reaction period are set to the times when the rotation of the rotator 61 by 180 degrees is finished (the rotator 61 is stopped), however, the times may be times when the rotator 61 by 180 degrees is started.

Further, in the above described embodiments, the rotation mechanism 60 is employed as a mechanism of alternately moving the droplet 47A within the PCR container 30 to the first region 36A and the second region 36B of the PCR container 30. However, various movement mechanisms other than the rotation mechanism 60 can be applied as long as the mechanism alternately moves the droplet 47A to the first region set at the denaturation temperature of the target nucleic acid in the PCR container and the second region set at the synthesis temperature of the target nucleic acid independent of the first region.

Furthermore, in the above described embodiments, as the regions to which the droplet 47A is to be moved in the PCR container, the first region set at the denaturation temperature of the target nucleic acid and the second region set at the synthesis temperature of the target nucleic acid independent of the first region are provided. However, for example, three regions may be provided as shown in Japanese Patent Application No. 2014-107844. That is, as the first region within the PCR container, a region set at the denaturation temperature of the target nucleic acid is provided. Further, as the second regions, two regions independent of each other are provided, and one region is set at an annealing temperature set as a temperature at which the annealing reaction in the synthesis reaction of the target nucleic acid progresses and the other region is set at an elongation temperature set as a temperature at which the elongation reaction of the target nucleic acid progresses. As described above, not only in the case of the above described embodiments in which the temperature changes in the cycle are at two stages of the denaturation stage and the synthesis stage, but also in the case of the three stages of the denaturation stage, the annealing stage and the elongation stage, the droplet 47A may be moved within the PCR container. Note that, even in the case where the temperature changes in the cycle are at the three stages, various movement mechanisms other than the rotation mechanism can be applied.

In the above described embodiments, the specific gravity of the droplet 47A to be housed within the PCR container 30 is set to be larger than that of the oil 37 filling the PCR container 30. However, the specific gravity of the droplet 47A may be set to be smaller than that of the oil 37. In this manner, the same effects as those of the above described embodiments may be exerted.

Further, in the above described embodiments, the PCR container 30 is filled with the oil 37. However, the oil 37 may be omitted as long as the droplet 47A moves within the PCR container 30 without being broken. That is, the oil 37 filling the PCR container 30 is not an essential component element.

In the above described embodiments, the nucleic acid amplification apparatus 50 including the heater on high-temperature side 65B and the heater on low-temperature side 65C is applied. However, a nucleic acid amplification apparatus other than the nucleic acid amplification apparatus 50 of the above described embodiments may be applied as long as a temperature gradient may be formed within the PCR container 30. For example, only the heater on high-temperature side may be provided and the heater on low-temperature side 65C may be changed to a cooler. Alternatively, the heater on high-temperature side and the heater on low-temperature side may be provided outside of the rotator 61. Alternatively, the part in which the heater on high-temperature side 65B is provided and the part in which the heater on low-temperature side 65C is provided may be reversed.

Further, in the above described embodiments, the heater for elution 65A is provided, however, the heater may be omitted. Note that it is desirable that the nucleic acid amplification apparatus 50 includes the heater for elution 65A because the release of the nucleic acid from the magnetic beads is promoted.

In the above described embodiments, the magnet movement mechanism 70 is provided, however, the mechanism may be omitted. In the case where the magnet movement mechanism 70 is omitted, for example, a worker may hold the magnet and moves the magnet along the tube 20. Note that it is desirable that the magnet movement mechanism 70 is provided because the movement velocity of the magnetic beads 7 as the solid-phase supports having the nucleic acid binding property may differ depending on the skill of the worker.

In the above described embodiments, the pressure mechanism 80 is provided, however, the mechanism may be omitted. In the case where the pressure mechanism 80 is omitted, for example, a worker may push the plunger 10 of the cartridge by hand. Note that it is desirable that the pressure mechanism 80 is provided because the amount of pressure for pushing the plunger 10 per unit time may differ depending on the skill of the worker.

In the above described embodiments, the fluorophotometer 55 is provided, however, the fluorophotometer 55 maybe omitted. In the case where the fluorophotometer 55 is omitted, the quantification of the amplification of the nucleic acid is impossible, but the amplification of the nucleic acid is possible.

The entire disclosure of Japanese Patent Application No. 2015-004833, filed Jan. 14, 2015 is expressly incorporated by reference herein.

1. A nucleic acid amplification method comprising:
a heating step of heating a first region of a container housing a droplet containing a template nucleic acid and a sample necessary for amplification of a target nucleic acid in the template nucleic acid to a denaturation temperature of the target nucleic acid and heating a second region different from the first region to a synthesis temperature of the target nucleic acid; and
an amplification step of repeating a cycle through a denaturation stage at which the droplet housed in the container is moved to and retained in the first region and a synthesis stage at which the droplet is moved to and retained in the second region at a plurality of times,
wherein, at the amplification step, periods of part of cycles of the plurality of the cycles are made shorter than periods of the other cycles.

2. The nucleic acid amplification method according to claim 1, wherein, at the amplification step, periods of the respective cycles following the time as a specified-numbered time counted from the first time are made shorter than periods of the respective cycles until the time as the specified-numbered time counted from the first time.

3. The nucleic acid amplification method according to claim 1, wherein, at the amplification step, periods of the respective cycles to the time as a specified-numbered time counted from the first time are made shorter than periods of the respective cycles of the times following the time.

4. The nucleic acid amplification method according to claim 1, wherein denaturation reaction periods of the denaturation stages in the part of cycles are made shorter than denaturation reaction periods of the denaturation stages in the other cycles, and
synthesis reaction periods of the synthesis stages in the part of cycles are not made shorter than synthesis reaction periods of the synthesis stages in the other cycles.

5. The nucleic acid amplification method according to claim 4, wherein a partial period of a period of the shortened amount of the denaturation reaction periods of the denaturation stages in the part of cycles made shorter than denaturation reaction periods of the denaturation stages in the other cycles is assigned to the synthesis reaction periods of the synthesis stages in the part of cycles.

6. A nucleic acid amplification apparatus comprising:
an attachment part to which a container housing a droplet containing a template nucleic acid and a sample necessary for amplification of a target nucleic acid in the template nucleic acid is attached;
a heater that heats a first region in the container attached to the attachment part to a denaturation temperature of the target nucleic acid and heating a second region independent of the first region to a synthesis temperature of the target nucleic acid;
a movement mechanism that moves the droplet from the first region to the second region or from the second region to the first region; and
a control section that controls the movement mechanism to repeat a cycle through a denaturation stage at which the droplet is retained in the first region and a synthesis stage at which the droplet is retained in the second region at a plurality of times,
wherein the control section makes periods of part of cycles of the plurality of the cycles shorter than periods of the other cycles.

7. The nucleic acid amplification method according to claim 2, wherein denaturation reaction periods of the denaturation stages in the part of cycles are made shorter than denaturation reaction periods of the denaturation stages in the other cycles, and
synthesis reaction periods of the synthesis stages in the part of cycles are not made shorter than synthesis reaction periods of the synthesis stages in the other cycles.

8. The nucleic acid amplification method according to claim 3, wherein denaturation reaction periods of the denaturation stages in the part of cycles are made shorter than denaturation reaction periods of the denaturation stages in the other cycles, and
synthesis reaction periods of the synthesis stages in the part of cycles are not made shorter than synthesis reaction periods of the synthesis stages in the other cycles.

9. The nucleic acid amplification method according to claim 7, wherein a partial period of a period of the shortened amount of the denaturation reaction periods of the denaturation stages in the part of cycles made shorter than denaturation reaction periods of the denaturation stages in the other cycles is assigned to the synthesis reaction periods of the synthesis stages in the part of cycles.

10. The nucleic acid amplification method according to claim 8, wherein a partial period of a period of the shortened amount of the denaturation reaction periods of the denaturation stages in the part of cycles made shorter than denaturation reaction periods of the denaturation stages in the other cycles is assigned to the synthesis reaction periods of the synthesis stages in the part of cycles.
